(19) United States
(12) Patent Application Publication
(10) Pub. No.: US 2026/0182908 A1
(43) Pub. Date: Jul. 2, 2026

(54) DETECTION OF RISK OR OCCURRENCE OF STROKE, ANEURYSM, OR BRAIN INFARCTION

(71) Applicant: NEUROVIGIL, INC., Moffett Field, CA (US)

(72) Inventor: Philip LOW, Moffett Field, CA (US)

(73) Assignee: NEUROVIGIL, INC., Moffett Field, CA (US)

(21) Appl. No.: 19/547,466

(22) Filed: Feb. 23, 2026

Related U.S. Application Data
(63) Continuation of application No. PCT/US2024/048707, filed on Sep. 26, 2024.
(60) Provisional application No. 63/586,793, filed on Sep. 29, 2023.

(51) Int. Cl. A61B 5/00; A61B 5/245; A61B 5/372; A61B 5/397; A61B 5/398

(52) U.S. Cl. CPC A61B 5/4818; A61B 5/245; A61B 5/372; A61B 5/397; A61B 5/398; A61B 5/4058; A61B 5/4809; A61B 5/4812; A61B 5/7235; A61B 5/746

(57) ABSTRACT

The present disclosure relates to a method and system for acquiring and analyzing physiological signals of a subject to detect risk or occurrence of stroke or brain infarction. A wearable wireless device together with one or more clusters of electrodes can be used to record the physiological signals during the sleep-wake cycle. The physiological signals may be utilized to perform sleep and wake analysis. In some instances, the physiological signals corresponding to each hemisphere of the brain may be compared, for example, using coherence, to quantify asymmetry across hemispheres and to identify possible strokes, silent brain infarcts (SBIs), or aneurysms. Biomarkers may be extracted based on sleep and wake analysis and coherence. The biomarkers may include an indication of obstructive sleep apnea or apnea risk, a reduced duration of slow wave sleep, or a presence of SBIs, or aneurysms. A prediction model may be used to generate a stroke risk score based on the biomarkers for early detection of potential individuals at risk of stroke.

### CROSS-REFERENCE TO RELATED APPLICATIONS

This application is a continuation of International Patent Application No. PCT/US2024/048707, filed on Sep. 26, 2024, which claims the priority to and the benefit of U.S. Provisional Application Number 63/586,793, filed on Sep. 29, 2023, entitled “Detection of Risk or Occurrence of Stroke of Brain Infarction”. The entire disclosures of the aforementioned applications are incorporated by reference herein in their entireties for all purposes.

### BACKGROUND

Stroke is a leading cause of death worldwide and is a major cause of serious disability for adults. Time is of the essence when treating neurodegenerative abnormalities such as stroke. For example, common treatments for stroke include administering a tissue plasminogen activator (which dissolves blood clots to increase blood flow) or removing a clot using an endovascular procedure. However, if these treatments are not delivered quickly, neurons will have already died due to lack of blood supply. Strokes can be categorized into two types: ischemic stroke and hemorrhage stroke, either of which can cause death of neurons and therefore lead to further (usually irreversible) brain damage or even death.

However, the stroke can be prevented through early diagnosis and timely interventions. Moreover, before the actual stroke, individuals usually experienced mini-strokes or transient ischemic attacks (TIAs) that may be caused by an aneurysm. Therefore, specialized systems that can automatically screen or identify individuals at risk of stroke or having aneurysm, preferably in home settings, long-term care, or in non-hospitalized settings, can be life-saving. Therefore, there is a demand for an improved system to accurately detect the stroke and/or predict a stroke risk of the individuals for an early detection of those individuals that may encounter a stroke in near future. Such systems may improve the quality of life of the individuals through stroke prevention, timely interventions, and may reduce financial burden on the healthcare systems and the individuals.

### SUMMARY

Some embodiments of the present disclosure relate to use of physiological signals of a subject to detect a risk or an occurrence of stroke in a brain of the subject. A computer-implemented method includes accessing physiological data of the subject that was collected by the physiological data acquisition assembly over a period of time. The physiological data acquisition assembly may be comprised of a sensing device and one or more clusters of electrodes. The sensing device may include an accelerometer and a gyroscope. The sensing device can be utilized to acquire, process and transmit signals from the one or more clusters of electrodes. The one or more clusters of electrodes may include electroencephalogram (EEG) electrodes, electromyography (EMG) electrodes, magnetoencephalography (MEG) electrodes, or electrooculogram (EOG) electrodes. Each cluster of the one or more clusters of electrodes comprises at least an active electrode. Other electrodes in each cluster may include a reference electrode, or a bias electrode. The physiological data may correspond to physiological signals (e.g., EEG, EMG, EOG, MEG) that were collected a night-time period, a rest period, or a plurality of previous night-time or rest periods.

A sleep architecture may be generated by analyzing the physiological data of the subject. For instance, generating the sleep architecture may include extracting a set of features based on a portion of the physiological data corresponding to each time interval of a plurality of time intervals within the period of time. The set of features may be associated with one or more frequency bands of the physiological signals corresponding to each time interval. The set of features may include one or more of Delta power, Gamma power, standard deviation, maximum amplitude, Gamma power/Delta power, time derivative of Delta, and time derivative of the Gamma power/Delta power. The set of features may further include features that are derived using component analysis (e.g., principal component analysis PCA, independent component analysis ICA) from a spectrogram or a normalized spectrogram of the one or more frequency bands of the physiological signals for the time interval.

Further, a state can be predicted for each of the set of features corresponding to each time interval. The state may correspond to any of one or more sleep stages or an awake state. The one or more sleep stages may include a rapid eye movement (REM) stage and one or more non-REM stages. In some instances, a sleep classification model may be used to predict the state. The sleep classification model may include supervised machine learning techniques such as decision trees, support vector machine (SVM), random forest, or neural networks that are trained on labeled sleep data. In some other instances, clustering techniques, for example, k-means clustering, hierarchical clustering or Gaussian mixture model (GMM) can be utilized to predict the state for each time interval. Based on the predicted state for each time interval, a sleep pattern, a relative frequency of each of the one or more sleep stages or the awake state, and a duration of each of the one or more sleep stages or the awake state may be determined. In addition, spectral and temporal fragmentations may also be calculated.

Further, one or more biomarkers may be determined based at least in part by using the sleep architecture of the subject. The one or more biomarkers may include an indication of apnea, an apnea risk, a change in sleep, or a change across hemispheres. In some instances, a first portion of the physiological data corresponding to a first hemisphere of a brain of the subject may be determined. Similarly, a second portion of the physiological data corresponding to a second hemisphere of the brain of the subject may also be determined. Afterwards, a coherence can be computed between the first portion and the second portion of the physiological data. The first portion and the second portion may correspond to a segment (or an epoch) of the physiological data (e.g., EEG signals) or may correspond to a sleep-awake cycle. Based on the apnea, the apnea risk, the change in sleep, and the coherence, a presence or likelihood of a hemorrhagic stroke, silent brain infarcts (SBIs), a transient ischemic stroke (TIA), an aneurysm, a brain tumor, or a traumatic brain injury (TBI) may be determined. Moreover, the one or more biomarkers may further include a presence of obstructive sleep apnea (OSA), a duration of OSA, an intensity of OSA, or a reduction in duration of slow wave sleep (SWS).

A stroke risk score of the subject may be predicted by using a prediction model based on the one or more biomarkers. In some embodiments, the stroke risk score is further predicted based on risk factors data comprising medical information and mobility metrics of the subject. The medical information may include a body mass index (BMI) value, an atrial fibrillation diagnosis, a diabetes mellitus diagnosis, a blood sugar level history, a blood pressure data, a family history of stroke or TIA, a family history of heart attack, or a blood cholesterol level. The mobility metrics may include an intensity of physical activity per week. The prediction model may be comprised of a machine learning model, including, a recurrent neural network (RNN), a transformer model, or a neural network.

Afterwards, determine whether a condition is satisfied based at least in part on the stroke risk score. For example, the condition can be such that whether the (predicted) stroke risk of the subject is greater than a specific threshold, whether the stroke risk score is indicative of high risk of future stroke, or whether the stroke risk score is medium and along with either apnea is present or SBI (including mini-stroke or TIA) is detected, and the like.

Furthermore, one or more actions may be triggered based on determining that the condition is satisfied. The one or more actions may include alerting the subject, alerting a caregiver or a clinician, or outputting a result that provides a basis for a recommendation or includes the recommendation to perform an evaluation or an intervening action to reduce the stroke risk score. The one or more actions may further include presenting the result and/or the stroke risk score of the subject on a computing device or transmitting the result to another device. The result may further include the sleep architecture, apnea results, interhemispheric comparisons (e.g., coherence), and stroke results to facilitate the clinician for further investigation and to decide a treatment plan for the subject.

In some embodiments, a system is provided that includes one or more data processors and a non-transitory computer-readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

Various embodiments are described hereinafter with reference to the figures. It should be noted that the figures are not drawn to scale and that the elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure.

FIG. 1 illustrates an example overview of a system to detect a risk or an occurrence of stroke of a subject in accordance with some embodiments of the present disclosure.

FIG. 2 illustrates an example placement of an adhesive film, electrodes, and a sensing device on a forehead of the subject in accordance with some embodiments of the present disclosure.

FIG. 3 illustrates an example pipeline to process physiological signals and to extract physiological features in accordance with some embodiments of the present disclosure.

FIG. 4 shows an example implementation of a sleep analyzer to perform sleep stage analysis in accordance with some aspects of the present disclosure.

FIG. 5 illustrates an example implementation of an apnea detection of the subject by leveraging output of the sleep analyzer, EEG data, and a possible one or more non-EEG sensor data in accordance with some embodiments of the present disclosure.

FIG. 6 shows an example illustration to process the EEG data of a left hemisphere and a right hemisphere to detect a stroke, mini-strokes, a transient ischemic attack (TIA), or silent brain infarcts (SBIs) in the brain of the subject.

FIG. 7 illustrates an example architecture to generate a stroke risk score of the subject by using a prediction model in accordance with some embodiments of the present disclosure.

FIG. 8 shows an example flowchart of the system for detecting the risk or the occurrence of stroke of brain infarction in the subject in accordance with some embodiments of the present disclosure.

FIG. 9 shows an example illustration of a computer system in which various embodiments of the present disclosure may be implemented.

### DETAILED DESCRIPTION

Disclosed embodiments of the present disclosure relate to a method and system for acquiring and analyzing physiological signals of a subject to detect a risk or an occurrence of stroke of brain infarction. The physiological signals of the subject may be collected during a sleeping state or resting state over a period of time by using a physiological data acquisition assembly. According to present disclosure, one or more biomarkers or subclinical stroke risk factors may be determined through analyses of the physiological signals. A prediction model may be used to generate a stroke risk score of the subject. According to some embodiments, a technical solution is provided in the present disclosure to a technical problem of early detection of individuals that are at risk of stroke or may have an aneurysm or SBIs, preferably in home settings (or even in hospitalized settings).

According to some aspects of the present disclosure, identification of subclinical risk factors may facilitate early and potentially more effective prevention measures for stroke, aneurysms, TBI, brain tumor and the like. One such potential risk factor is silent brain infarction. The term ‘silent brain infarcts (SBIs)’ may be used to describe cerebral infarcts or brain infarcts that can be seen on brain computed tomography (CT) or magnetic resonance imaging (MRI) without any corresponding stroke episode. The SBIs may represent areas of brain damage (e.g., an area of necrosis or tissue death) that occur due to a lack of blood flow (ischemia). SBIs are often discovered incidentally during imaging studies, such as MRI or CT scans, performed for other reasons. The subjects or individuals usually do not experience any noticeable symptoms and that is why SBIs are termed ‘silent’. Although asymptomatic, SBIs can be associated with cognitive decline, especially in older adults, and may increase the risk of future strokes or dementia. A direct potential sequela of SBIs is a symptomatic stroke. Approximately, 25% of elderly people with an age greater than 80 years may have more than 1 SBIs. The prevalence of SBIs can greatly exceed the prevalence of the symptomatic stroke. It has been estimated that every symptomatic stroke may be preceded by more than 10 SBIs. Furthermore, SBIs are associated with future incidents of cognitive decline and stroke. Hence, a system that can detect SBIs easily without the need to schedule CT or MRI can be of great benefit and effective in stroke prevention.

Similarly, ischemic strokes may be preceded by one or more “mini-strokes,” also called transient ischemic attacks (TIAs). A TIA, often referred to as a mini-stroke, is a temporary episode of neurological dysfunction caused by a brief period of insufficient blood flow to the brain. TIAs may last for a few minutes or up to 24 hours and are often a warning sign that a stroke may occur in future. Common symptoms may include sudden numbness or weakness on one side of the body, difficulty speaking, sudden confusion, or vision problems. SBIs, mini-strokes, TIAs may be caused due to multiple reasons including vessel disease such as aneurysm. According to disclosed embodiments, SBIs, TIAs, or mini-strokes may be detected based on the physiological data (e.g., EEG signals) and the subject may be recommended for detailed checkup by the clinician before the aneurysm bursts.

Furthermore, some other subclinical risk factors of stroke can be obstructive sleep apnea (OSA) and a reduced duration of slow wave sleep (SWS). Reduced SWS and severe OSA may be associated with higher burden of white matter abnormalities in predominantly cognitively unimpaired older adults, which may contribute to greater risk of cognitive impairment, dementia, and stroke. OSA may also be disproportionately linked to stroke. According to some aspects of the present disclosure, the physiological signals (e.g., EEG signals) or the physiological data of the subject may be utilized to perform sleep analysis or sleep stage analysis. The sleep stage analysis may categorize each epoch or segment of the EEG signals into one of several predefined sleep stages, including awake, REM, and Non-REM stages such as SWS, stage I, and stage II. The sleep stage analysis may generate outputs, arousal or micro-arousal detection results, microsleep detection results, a sleep pattern, relative frequency and duration of each stage of the several predefined stages, a sleep score, or a hypnogram. OSA may often cause transient arousals and can be detected as sudden changes in the EEG signals. Therefore, based on the sleep analysis additional risk factors or subclinical risk factors such as OSA, or reduced SWS may be determined.

During resting or sleeping states, the EEG data or signals exhibit bilateral symmetry. The strokes are usually unilateral, hence, a decay or deterioration in coherence across hemispheres may indicate the symptomatic stroke, mini-strokes, TIA, or SBI. Interhemispheric comparisons such as using statistical techniques, clustering techniques, distance metrics, or coherence may provide valuable insights into the functional connectivity and synchronization of brain activity across different regions and hemispheres. In some instances, the physiological signals corresponding to each hemisphere of the brain may be compared based on the Interhemispheric comparisons, to quantify asymmetry across hemispheres and to detect a presence or likelihood of neurodegenerative abnormality. Hence, biomarkers (or subclinical risk factors) may be extracted based on the sleep analysis and the interhemispheric comparison results. The biomarkers may include a detection or severity of OSA, a reduced duration of SWS, or a presence of SBIs. After detecting the presence of the neurodegenerative abnormality, disclosed techniques in the present disclosure may further detects or identify the presence or likelihood of the hemorrhagic stroke, mini-stroke, TIA, SBIs, aneurysms, brain tumor, or TBI, based on the one or more biomarkers such as apnea, the apnea risk, the change in sleep, and the interhemispheric comparison results.

Further, a prediction model may be used to generate a stroke risk score based on the biomarkers for early detection of potential individuals at risk of stroke. The prediction model can include a machine-learning model, such as a recurrent neural network (RNN), a long short-term memory (LSTM) model, a transformer model, neural networks (NNs), multi-value prediction algorithm, deep learning models, or regression techniques. The stroke risk score can be a number (e.g., 0-10), a binary value, or a category (e.g., mild, moderate, or high risk).

In some instances, the prediction model may utilize, in addition to biomarkers, other risk factors data, for example, clinical risk factors or non-EEG based risk factors. The risk factors may include but are not limited to a body mass index (BMI) value, an atrial fibrillation diagnosis, a diabetes mellitus diagnosis, a blood sugar level history, a blood pressure data, a family history of stroke or TIA, a family history of heart attack, or a blood cholesterol level. The mobility metrics may include, for example, an intensity of physical activity per week.

After predicting the stroke risk score, one or more actions may be triggered based on determining that a condition is satisfied. The one or more actions may include alerting the subject, alerting a caregiver or a clinician, or outputting a result or data that provides a basis for a recommendation or includes the recommendation to perform an intervening action such as a medical evaluation, and a possible MRI to reduce the stroke risk score. The one or more actions may further include presenting the result and/or the stroke risk score of the subject on a user device (e.g. smartwatch, smartphone) or transmitting the result to another device. The result may further include the biomarkers, the sleep analysis results, apnea results, comparison and detection results to facilitate the clinician for further investigation and to decide a treatment plan for the subject.

In some instances, the neurodegenerative abnormality detection system includes a physiological data acquisition assembly that is configured to obtain physiological data associated with the brain of the subject. The physiological data acquisition assembly may include a sensing device and one or more clusters of electrodes. Each cluster of the one or more clusters of electrodes includes at least one active electrode. The one or more clusters of electrodes may further include a reference electrode or a ground electrode. The electrodes can include electroencephalogram (EEG) electrodes, electromyography (EMG) electrodes, magnetoencephalography (MEG) electrodes, electrooculogram (EOG) electrodes, electrocardiogram (EKG), and the like. The electrodes may be dry contact electrodes, dry non-contact (capacitively coupled) electrodes, or wet contact electrodes.

The physiological data acquisition may further include a transmitter and potentially a receiver (which may be a single transceiver). The transmitter can be configured to communicate data corresponding to signals recorded by the electrodes to a computing device that is part of the neurodegenerative abnormality detection system. The computing device may be a device operated by the subject, by a medical provider associated with treating the subject, or an entity facilitating medical monitoring or treatment for the subject. Such communication can occur using any of a variety of commercially available protocols, such as a wireless network, including a short-range connection (e.g., a Bluetooth, Bluetooth low energy (BTLE), or ultra-wideband connection) or over a WiFi network, such as the Internet, etc. In some instances, a receiver is configured to receive an instruction or request from a computing device, such as an instruction to begin recording signals or a request to send data to the computing device.

Furthermore, the physiological data acquisition assembly can be implemented as a wearable device, for example, a sensing patch. The sensing patch may be comprised of an adhesive film, the electrodes, and the sensing device. In some embodiments, the electrodes, along with connecting wires (or electrode leads), may be implemented using a flexible printed circuit board (PCB) and can be attached to the subject using an adhesive material (e.g., the adhesive film) or some type of gel for better signal acquisition. The sensing device may be adhered to the flexible printed circuit board and can be connected to the electrodes through PCB traces. In some other instances, the sensing device and the electrodes may be implemented jointly on the flexible PCB to develop the sensing patch. Moreover, the electrode structure (e.g., the number of electrodes or channels, their locations, size etc.) on the flexible PCB can be controlled during the fabrication process. The sensing patch may include at least one active electrode and a microprocessor (e.g., inside the sensing device) configured to transmit a signal collected by the active electrode or a processed version thereof.

In some instances, the physiological data acquisition assembly may include a wearable component such as a head harness, one or more straps, one or more bands, a hat, a helmet, or a cap, where each of multiple electrodes are positioned in locations that are expected to align with specific brain regions when the sensing device is being worn. The wearable component may have receiving components (e.g., an opening to receive a sensing patch or an electrode). The wearable component can facilitate ensuring that the electrodes, or the adhesive films are positioned at target positions on a subject. In addition, instructions may be provided to a subject to indicate where the electrodes or the adhesive films are to be placed.

The physiological data acquisition assembly may include a processing component that may perform initial processing using the signals recorded by the electrodes. Such processing may occur using execution of software code and/or using hardware elements. The initial processing may include amplification of the signals recorded by the electrodes, determining a differential signal, applying a filter (e.g., to remove signals around 50 Hz or 60 Hz depending on the geographical region or to focus on frequency bands of interest), and/or down sampling the signals. A differential signal may be determined by subtracting a signal from one electrode. For example, a signal from a reference electrode may be subtracted from a signal from an active electrode or a signal from a first active electrode may be subtracted from a signal from a second active electrode.

In some instances, one or more initial processing actions may instead or additionally be performed at the computing device to which the signals are transmitted. The computing device may include a mobile device (e.g., a smart phone), a tablet, a laptop, a desktop computer, a computer server, and the like.

Various disclosures herein relate to using a physiological data acquisition assembly to improve techniques for predicting whether a given individual is at risk of experiencing or has experienced a stroke, aneurysms, or brain infarction. In other instances, the disclosed techniques can be used to assess the efficacy of interventions or to monitor recovery of a subject who had experienced a neurodegenerative abnormality such as a stroke.

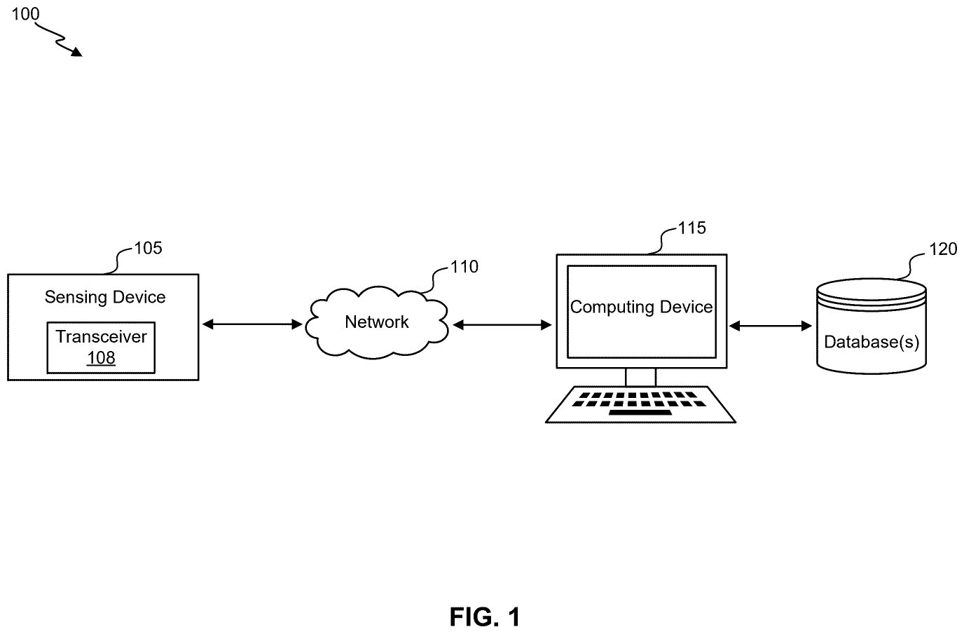

FIG. 1 illustrates an example overview of a system to detect risk or occurrence of stroke in accordance with some embodiments of the present disclosure. Exemplary system 100 comprises a sensing device 105, a network 110, a computing device 115, and one or more database(s) 120. The sensing device 105 may include a transceiver 108 to communicate with the computing device 115. The sensing device 105 may be connected to the computing device 115 through the network 110. The transceiver 108 can be configured to communicate physiological data recorded by the sensing device 105 to the computing device 115 that is part of the neurodegenerative abnormality detection system.

The computing device 115 may be a device operated by the subject, by a clinician or the medical provider associated with treating the subject, or an entity facilitating medical monitoring or treatment for the subject. The computing device 115 may include a mobile device (e.g., a smartphone), personal digital/data assistants (PDA), a tablet, a laptop, a desktop computer, a computer server, and the like. In some instances, the transceiver 108 and/or the sensing device 105 can be configured to receive an instruction or request from the computing device 115, such as an instruction to begin recording signals or a request to send data to the computing device 115. Moreover, the communication between the sensing device 105 and the computing device 115 can occur using the network 110, which can be a wireless network based on a commercially available communication protocols, for example, Bluetooth, Bluetooth low energy, ultra-wideband connection, or WiFi network, such as the Internet, etc. The network 110 may include, internet, an intranet, a cellular network, a wired LAN (local area network), a wireless LAN (WiLAN), a WAN (wide area network), a MAN (metropolitan area network), a PSTN (public switched telephone network), and other types of communications networks. The network 110 may further include communication devices such as one or more gateways, routers, or bridges. The network 110 may be any type of network familiar to those skilled in the art that can support data communications using any of a variety of available protocols, including without limitation TCP/IP (transmission control protocol/Internet protocol), SNA (systems network architecture), IPX (internet packet exchange), AppleTalk®, and the like.

According to some embodiments, the physiological data acquisition assembly may include the sensing device 105, different types of electrodes, and/or wearable components. The different types of electrodes may include but are not limited to dry electrodes, wet electrodes, self-adhesive conductive electrodes, electrodes with snap connectors, the EEG electrodes, the EMG electrodes, the EOG electrodes, the MEG electrodes, and the like.

In some embodiments, the sensing device 105 is configured to acquire, record, process, and transmit physiological data associated with the brain of the subject. In some instances, the physiological data comprises electrical activity of the brain and can be recorded using EEG electrodes attached to the scalp or on the forehead of the subject. The sensing device 105 may be configured with at least one active electrode and a reference electrode. The active electrode acts as a primary sensor that detects the electrical activity that is directly or indirectly generated by the neuronal firing in the brain and nervous system. The active electrodes pick up the electrical activity generated by the brain and nervous system and further, transmit these signals to the sensing device 105 for initial processing (e.g., signal amplification, analog to digital conversion, denoising etc.) and analysis. The reference electrode provides a baseline or common point of comparison for the active electrodes. The sensing device 105 may further include a ground (or bias) electrode. The ground or bias electrode can be placed behind the ear of the subject or can also be placed along with the active or reference electrodes. The bias electrode functions to stabilize the baseline or electrical potential of an EEG system and reduces noise or interference from external electrical sources. In some instances, the physiological data acquisition assembly may omit the ground or bias electrode and may utilize the reference and active electrodes for data recording. This is because modern differential amplifiers can be designed to operate without a dedicated ground electrode by using a virtual ground created internally by the amplifier circuitry.

In some other embodiments, the sensing device 105 may be configured to record and stored physiological data in encrypted format and to transmit wirelessly to a remote center or the computing device 115 for further display, storing, processing, and analysis. In another aspect of the present disclosure, the recorded and stored physiological data may be transmitted wirelessly in real-time to the computing device 115 including a cellular telephone, smartphone, tablet, and/or computer. The recorded and stored physiological data may also be transmitted directly to a computer, cellular telephone, smartphone and/or tablet via universal serial bus (USB) transfer capabilities incorporated within the sensing device 105.

During physiological data acquisition, an initial or pre-amplification may be performed at or near the electrodes to reduce noise. For example, the electrode snap connector assemblies may include a noise reducing or cancelling filter at the electrode connection level to reduce any electrical noise that may be picked up by the lead wires. To further improve the physiological data acquisition process, the sensing device 105 or the electrode snap connector assemblies can be configured to continuously monitor electrode impedance and may include lights indicative of the current status of the integrity of the electrode connection with the scalp or forehead. The sensing device 105 may comprise hardware and software components (e.g., firmware or signal processing code) and can be used to perform initial processing. The initial processing may include amplification of the signals recorded by the electrodes, determining a differential signal, applying a filter (e.g., to remove signals around 50-60 Hz or to focus on frequency bands of interest), and/or downsampling the signals. A differential signal may be determined by subtracting a signal from one electrode. For example, a signal from a reference electrode may be subtracted from a signal from an active electrode, or a signal from a first active electrode may be subtracted from a signal from a second active electrode.

The sensing device 105 may further include a battery power component that can include a rechargeable small form factor, high-capacity battery. In some instances, the battery power component may include a disposable battery. The sensing device 105 may further include a memory, a processor, and the transceiver 108 for transmission, for example, to the computing device 115, or the one or more database(s) 120. The sensing device 105 includes a power supply and recharging circuitry for receiving power through an electrical power cord and alternating current (AC) unit. The electrical power cord is coupled to the sensing device 105 for recharging the small form factor, high-capacity battery through a port, which may be but is not limited to USB, D-subminiature (DB)-25, or the like. The sensing device 105 includes a power on and off function for preserving the power supply of the small form factor, high-capacity battery when not in use. The sensing device 105 may also include power on and off indicator lights indicative of the current status of the sensing device 105. In some instances, the sensing device 105 may be recharged through a USB connection to a computer. In some other instances, the sensing device 105 may be recharged wirelessly.

The exemplary system 100 may further include the one or more database(s) 120 for storing and future processing of data (e.g., EEG signals of the subject). The physiological data of the subject may be stored with metadata. The metadata may include subject information, type, and placement location of each electrode etc. In some instances, the sensing device 105 may transmit the EEG signals with metadata or in an order to indicate which EEG signals correspond to electrodes that were to be placed on contralateral sides of the subject. In some instances, the computing device 115 may associate various signals from active electrodes with different sides of the subject but does not specifically determine or predict which signals specifically correspond to a “left” side or hemisphere or a “right” side or hemisphere. One or more database(s) 120 may be elemental to a memory system on the computer or in secondary storage such as a hard disk, floppy disk, optical disk, or other non-volatile mass storage devices. In addition, the computing device 115 may be used to execute signal processing techniques or algorithms on the physiological data (previously recorded signals or real-time signals) and to store the results in the one or more database(s) 120. In one embodiment, the sensing device 105 may include a storage, and in another embodiment, the sensing device 105 may also have processor(s) for computation.

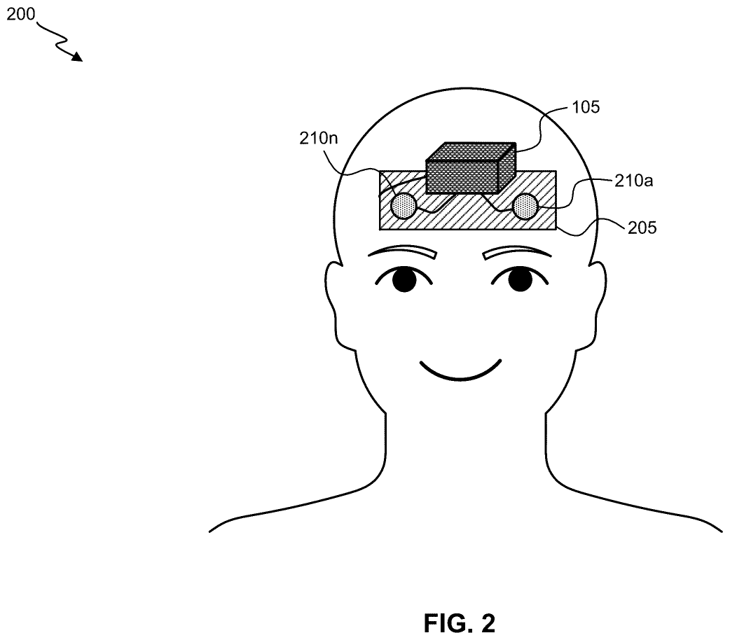

FIG. 2 illustrates an example placement 200 of an adhesive film 205, electrodes, and the sensing device 105 on a subject forehead in accordance with some embodiments of the present disclosure. According to the example placement 200, two or more electrodes 210a-n and the sensing device 105 may be adhered to the adhesive film 205 to capture the physiological signals of the left hemisphere and the right hemisphere. In some instances, the adhesive film 205 can be a single long adhesive film that is attached to the forehead of the subject. The two or more electrodes 210a-n may include the active electrodes, the reference, and the bias electrodes. The electrodes can be placed close to each other on the sensing patch. In some instances, a bias electrode may be attached to the ear (e.g., ear lobe or back side of the ear) of the subject using an electrode lead. In some other instances, the bias electrode can be skipped and the two or more electrodes 210a-n may only include the active electrodes and the reference electrodes. In some instances, the sensing device 105 is not on the head.

In some embodiments, the adhesive film 205 may be comprised of stretchable materials. In some instances, the two or more electrodes 210a-n, along with connecting wires (or electrode leads), may be implemented using a flexible printed circuit board (PCB) and can be attached to the subject using an adhesive material, headband, glasses (or goggles), or some type of gel for better signal acquisition. The sensing device 105 may be adhered on the flexible printed circuit board and can be connected to the two or more electrodes 210a-n through PCB traces. In some other instances, the sensing device 105 and the two or more electrodes 210a-n may be implemented jointly on the flexible PCB that may act as a singular sensing patch. Moreover, the electrode structure (e.g., the number of electrodes or channels, their locations, size etc.) on the flexible PCB can be controlled during the fabrication process. The physiological data or signals may be acquired during a sleeping state, or a resting state.

The collective composition of the adhesive film 205, the two or more electrodes 210a-n, and the sensing device 105 may be referred to as the sensing patch. The sensing patch may have a surface or the adhesive film 205 that extends across a length and width dimension. An adhesive material may be disposed across part or all of the surface or the adhesive film 205 (e.g., across part or all of one or more edges of the surface). The length may be (for example) less than 10 cm, less than 8 cm, less than 6 cm, less than 4 cm, less than 2 cm, etc. The width may be (for example) less than 10 cm, less than 8 cm, less than 4 cm, etc. The length may be (for example) greater than 0.5 cm, greater than 1 cm, greater than 2 cm, greater than 4 cm, etc. The width may be (for example) greater than 0.5 cm, greater than 1 cm, greater than 2 cm, greater than 4 cm, etc. The length may be (for example) between 0.5-10 cm, between 1-6 cm, between 2-4 cm, between 2-8 cm, between 2-8cm and/or between any other semi-closed or closed range having a threshold disclosed herein. The width may be (for example) between 0.5-10 cm, between 1-6 cm, between 2-4 cm, between 2-8 cm, between 2-8cm and/or between any other semi-closed or closed range having a threshold disclosed herein.

In some other embodiments, two or more sensing patches may be used to acquire physiological signals from the left hemisphere and the right hemisphere of the brain of the subject. For example, two sensing patches can be used and may be attached at two different locations such as one on each side of the brain or on the forehead. In some instances, disjoint adhesive films can be used at different locations. Two or more electrodes 210a-n may be adhered to each of the disjoint adhesive films. Similarly, each adhesive film may be connected with the transceiver 108 or the sensing device 105. In some other instances, the sensing patch may incorporate a range of physiological sensors such as EEG, EOG, EMG, and MEG sensors. These sensing patches can be strategically placed on various parts of skull to capture a comprehensive set of physiological data. Each sensing patch may operate independently yet communicates with the computing device 115, providing continuous monitoring even if one patch experiences a temporary failure or interference. For example, one sensing patch may be focused on monitoring neural signals (e.g., EEG, MEG), while another may track eye movements (EOG) and muscle activity (EMG).

In some instances, the physiological data acquisition assembly may include wearable components in addition to or instead the adhesive film(s). The wearable components may include one or more straps, one or more bands, or a cap and may have receiving components (e.g., an opening to receive a patch or an electrode). The wearable components can facilitate ensuring that the two or more electrodes 210a-n and/or films are positioned at target positions on a subject. In some cases, the sensing device 105 can be housed in the wearable components such as a head harness, for example, in accordance with the head harness and wireless EEG monitoring system, which is disclosed in U.S. application Ser. No. 17/214,574, filed on Mar. 26, 2021, which is hereby incorporated by reference in its entirety for all purposes. The head harness comprises of straps, and fasteners (e.g., Velcro, hook, button, etc.) for custom fit, adjustment, and comfort of the subject. The head harness may further include plurality of slots for attaching electrodes or electrode snap connectors at specific positions. For example, the bias electrode and the reference electrode may be attached behind the left and right ear and the active electrodes on the forehead of the subject.

In addition, instructions may be provided to the subject that indicate where the two or more electrodes 210a-n, one or more adhesive films, or the sensing patches are to be placed. For example, a drawing or photograph may be provided that show where each of one or more adhesive films are to be adhered on a subject's head (e.g., with a first film on a left side of the forehead and a second film on a right side of the forehead or with an elongated film positioned across the forehead of the subject).

The physiological data or signals may be acquired, for example, during a sleeping state, or a resting state. The physiological data may be collected during a night-time period, a rest period, or a plurality of previous night-time or rest periods. Typically, the EEG signals from the right hemisphere and the left hemisphere exhibit patterns of synchronization and similar power in different frequency bands particularly during sleeping or resting states for a healthy subject. This phenomenon is also called bilateral symmetry in EEG signals. Bilateral symmetry is a characteristic feature of resting or sleep states, reflecting synchronized brain activity between the two hemispheres.

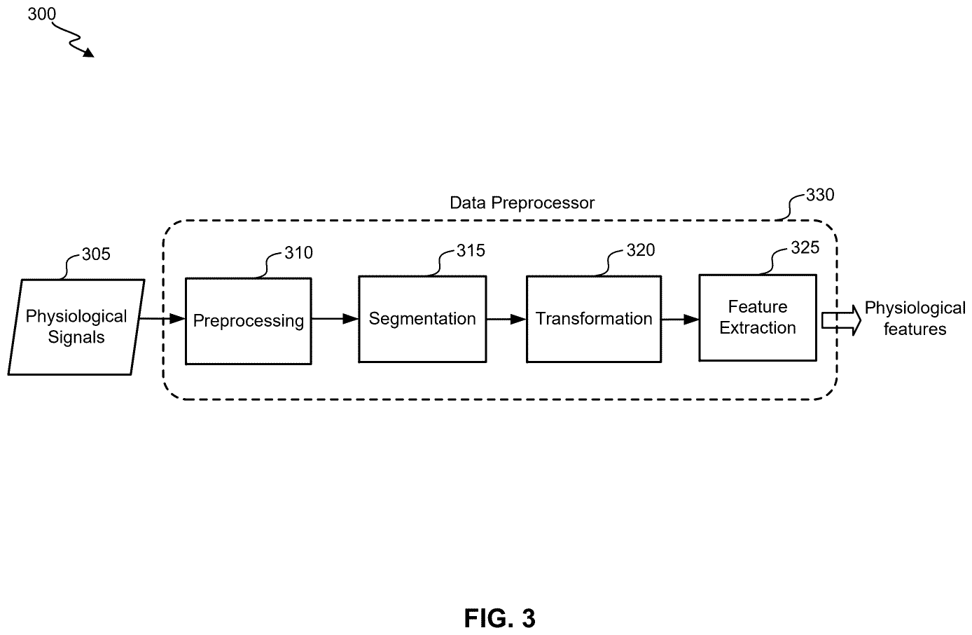

FIG. 3 illustrates an example pipeline 300 to process the physiological signals 305 and to extract physiological features in accordance with some embodiments of the present disclosure. After receiving the physiological signals 305, for example, from the sensing patch or the one or more database(s) 120, further processing and analysis can be performed on the computing device 115 as illustrated in FIG. 3. The physiological signals 305 (e.g., EEG signals) may be processed using a data preprocessor 330. The data preprocessor 330 includes modules such as a preprocessing 310, a segmentation 315, a transformation 320, and a feature extraction 325.

Physiological signals 305 may be processed to remove noise and other signal artifacts at preprocessing 310. During preprocessing 310, the physiological signals 305 may optionally be treated for removing artifacts, where an artifact refers to any part of the physiological signals 305 that misrepresents the data intended to be received. These artifacts may occur due to e.g., muscle activities such as jaw clenching or head movements causing high-frequency noise, periodic disturbances caused by electrical activity of the heart, or other environmental artifacts such as electromagnetic interferences, thereby impacting the accuracy of recorded physiological data. These artifacts can be removed from the physiological signals 305, for example, by automatically filtering out the physiological signals 305 via a filtering (e.g., direct current (DC) filtering), ICA, or data smoothing technique.

The physiological signals 305 can also be pretreated with component analysis, i.e., by decomposing the physiological signals 305 into independent components, identifying and removing artifacts based on the spatial and temporal characteristics. Physiological data artifacts may also be removed by estimating the artifact subspace using methods, e.g., principal component analysis (PCA) and projecting the physiological signals 305 onto orthogonal subspace for artifacts removal. In other instances, template matching may be performed that may identify and remove known artifact patterns by comparing the physiological signals 305 with predefined templates. Additionally, wavelet transform may be applied that decomposes the physiological signals 305 into different frequency components and removes artifacts in specific frequency bands.

After preprocessing 310, segmentation 315 may be performed on the physiological signals 305 (or EEG signals) that splits the signals (or continuous signals) into multiple time series increments (also referred herein as epochs) of similar or varying lengths. During segmentation 315, the time series increments or epochs may be segmented further into different sections using a scanning window, where the scanning window defines different sections of the time series increment (or epoch). The scanning window can move via a jumping window, resulting in non-overlapping sections or segments. For example, a one-hour epoch or time series increment of the physiological signals 305 can be scanned or segmented in increments of 1 minute (i.e., a scanning window of 1 minute), thus resulting in 60 disjoint or non-overlapping sections of the one-hour epoch. The scanning window can use a sliding window, where sections (or segments) of the sliding window may have overlapping time series sequences. For example, the one-hour epoch of the EEG signals can be scanned with a 1-minute scanning window that begins every 30 seconds (i.e., a sliding window of 30 seconds), thus resulting in a 1-minute scanning window that overlaps by 30 seconds. Alternatively, a whole time series of the EEG signals may correspond to an epoch.

The segments of the physiological signals 305 (e.g., which can include a differential EEG signal and/or a preprocessed EEG signal) can be transformed from a time domain to a frequency domain by transformation 320 module. For this purpose, power spectrum may be calculated e.g., by calculating power spectral density of each segment of the physiological signals 305 (e.g., the EEG signals). The power may be calculated by different techniques such as multi-taper transform, Fourier transform, or wavelet transform. In some instances, for each segment of the EEG signal and for each hemisphere, one or more normalizations may be applied in the time domain and/or in the frequency domain by transformation 320 module (e.g., in accordance with the SPEARS algorithm, which is disclosed in U.S. application Ser. No. 11/431,425, filed on May 9, 2006, which is hereby incorporated by reference in its entirety for all purposes). The EEG signal may be adjusted to account for differences in power by performing normalization. For example, normalization may be performed by weighing the spectral power of one or more segments (or time intervals) across time. The normalized power of each segment or time interval at one or more frequencies across time may help determining appropriate frequency windows for extracting information. Such normalization can reveal low power and statistically significant shifts in power at one or more frequencies bands. The frequency bands may include a band corresponding to Delta band, Theta band, Gamma band, Alpha band, Beta band, or any other frequency range.

The physiological signals 305, for example, the EEG signals may be characterized by different frequency bands associated with specific cognitive and physiological states. For example, the Delta band typically ranges from 0.5 Hz to 4 Hz and is characterized by slow waves with high amplitudes. Deep sleep such as Stage 3 of non-REM sleep that supports restorative processes may be associated with the Delta band. Similarly, the Theta band, which may range approximately around [4-8] Hz, comprises moderate frequencies and amplitude. Light sleep such as Stage 1 and 2 of non-REM sleep, drowsiness, meditation, or similar states may be associated with the Theta band. Alpha band may range approximately around [8-12] Hz and may characterize moderate frequencies with lower amplitudes than Delta and Theta band. Stage 2 of sleep can be characterized by sleep spindles, which typically occur in the [12-15] Hz frequency range. Various states such as relaxing, wakefulness with closed eyes, may be associated with Alpha band. Additionally, Alpha band may facilitate the transition between wakefulness and sleep. Followed by Alpha, Beta band approximately ranging from [12-30] Hz may be characterized by higher frequency with lower amplitude that may be associated with active thinking, focus, wakefulness, or similar activities. The frequency band with relatively higher frequencies, Gamma, approximately ranging [30-100] Hz may be characterized by high frequencies of EEG signals with low amplitude. Gamma band may be associated with high-level information processing and perception such as rapid eye movement sleep that may be characterized by vivid dreaming and high brain activity resembling wakefulness. In some instances, when the subject is alert and engaged in a task, gamma activity increases, enhancing the brain's ability to focus, process information rapidly, and maintain attention. which can be elemental for complex cognitive functions, such as problem-solving, memory recall, and conscious awareness. Increased gamma activity can often be observed when the subject appears fully attentive or deeply involved in tasks requiring high-level thinking and concentration. By processing these spectral characteristics of spectral bands, brain activity labels (e.g., various sleep stages, resting state, wakefulness, etc.) may be assigned to segments of the EEG signals.

Among these frequencies, one or more frequency bands can be revealed and utilized for further analysis. Feature extraction 325 may be performed on each segment of the physiological signal. Therefore, one or more features may be defined, which may include or be based on the power (or normalized power) in a transformed signal at each of one or more frequency bands. The one or more features may include a statistic that is determined based on one or more power values or weighted power values. For example, a feature may include a maximum or minimum power (or normalized power) in a spectrum corresponding to a segment, or a standard deviation (across frequency bands) of power, etc. As another example, a feature may include a standard deviation of power values associated with a given frequency band (or weighted power values) across segments. As yet another example, a feature may include a z-score, which can include a normalized unit that reflects the amount of power in the signal, relative to the average of the signal. The z-scores can be converted into mean deviation form, by subtracting the mean from each score. The scores can then be normalized relative to standard deviation. The z scored normalized units can have standard deviations that are equal to unity.

Features may be calculated epoch-wise by using each of the one or more epochs of data. As one illustration, features may be defined to include normalized power in low frequency bands (e.g., Delta band, Theta band, Alpha band), normalized power in a high frequency band (e.g., Gamma band), standard deviation of normalized power values across frequency bands in an epoch, a maximum normalized power value for the epoch, and the like. In addition, derived features can be generated based on the information (or normalized features) calculated for each of the one or more epochs of data. The derived features may include but not limited to Gamma power/Delta power, Gamma power/Alpha power, time derivative of Delta, time derivative of Gamma power/Delta power, time derivative of Gamma power/Alpha power. Time derivatives can be computed over preceding and successive epochs. Afterwards, the derived features can then be normalized across the one or more epochs. A variety of data normalization techniques can be conducted including z-scoring, min-max scaling, quantile transformation, log transformation and other similar techniques. In some instances, normalization is performed by z-scoring that is a statistical technique to standardize the range of independent variables (or features). It may involve transforming the features such that the features have a mean of zero and a standard deviation of one. By applying z-scoring, different derived features of the spectral power data such as Delta power and Gamma power/Delta power may be scaled to a common range, thus eliminating biases.

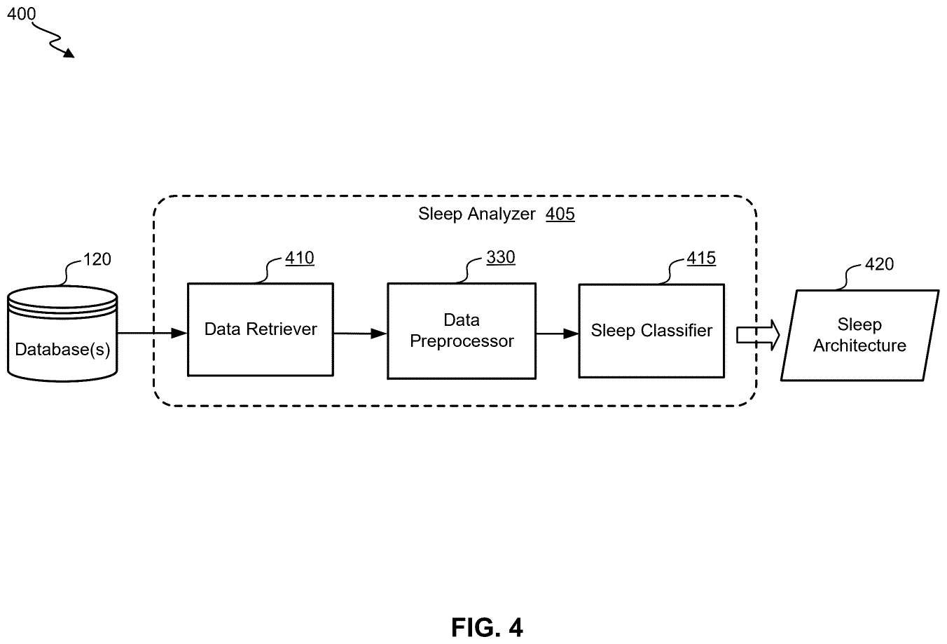

FIG. 4 shows an example implementation of a sleep analyzer 405 to perform sleep stage analysis in accordance with some embodiments of the present disclosure. The one or more database(s) 120 may be directly accessible by the sleep analyzer 405. The sleep analyzer 405 may assess recent sleep patterns, quality and/or duration of sleep of the subject by accessing the physiological data of one or more prior nights, or rest periods from the one or more database(s) 120. The sleep analyzer 405 may include a data retriever 410, the data preprocessor 230, and a sleep classifier 415. In some instances, the sleep analyzer 405 may analyze the physiological data from a resting period or an awake state to evaluate and interprets a level of engagement and attentiveness of the subject in an activity, a task, or an environment. In some other instances, neural signals and/or eye gazes may be used to predict the extent to which a subject is attending to a situation during wakefulness.

In some instances, the data retriever 410 may retrieve or obtain the physiological data of the subject from the one or more database(s) 120. In some other instances, the data retriever 410 may receive the data in real-time or near real-time while the subject is sleeping, resting, or awake, for example, from the sensing patch to further analyze it at the computing device 115. Once the physiological data and the metadata are retrieved, the data may be transmitted to the data preprocessor 330. The metadata may include the recording duration, recording date, time, electrodes placement or positions, and the like. The data preprocessor 330 may further prepare the physiological data for subsequent analysis. The data preprocessor 330 may clean the data to remove noise or artifacts and may segment the data into time windows or epochs (e.g., 30 secs, 2 min, 5 min, etc.). These segments can then be transformed as appropriate, and features may be extracted, highlighting important physiological indicators relevant to sleep stages, which may be utilized in further downstream analysis.

Afterwards, the preprocessed physiological data may be fed into the sleep classifier 415. The sleep classifier 415 may categorize each epoch or segment into one of several predefined sleep stages, including awake, REM, and non-REM stages such as SWS, stage I, and stage II. Awake can be further classified into quiet wakefulness and active wakefulness stages. The sleep classifier 415 may also detect microarousals and/or microsleeps, and specific landmarks. The sleep classifier 415 may assess parameters, such as the duration of each sleep stage, transitions between stages, and the overall architecture of the sleep that may facilitate in detection of pathological conditions.

In some embodiments, a sleep stage or state (including an awake state) may be assigned for each segment of the physiological signals 305 (or the physiological data) in accordance with U.S. application Ser. No. 11/431,425, which is hereby incorporated by reference for all purposes. Furthermore, the sleep pattern, the relative frequency, and the duration of each of the one or more sleep stages or the awake state can also be determined using the sleep classifier 415. The sleep classifier 415 may utilize a machine learning model. The machine learning model may include but is not limited to regression techniques (e.g., linear regression, polynomial regression), or classification techniques such as decision trees, random forests, support vector machines, neural networks, or deep learning models. The training of these models is typically performed using large, labeled datasets obtained from synthetic or augmented data, public sleep datasets or from Polysomnography studies. The standard datasets for sleep pattern analysis may include EEG, EOG, EMG, and often additional channels such as ECG or EKG. The physiological signals 305 which may include pre-processed physiological data may further be used to create labeled datasets for training models. The dataset for the training of the sleep classifier 415 may contain epochs of physiological data labeled with the correct sleep stage, which may be determined by experts through manual scoring or may be through automatic scoring. In some instances, unsupervised techniques such as clustering techniques e.g., k-means clustering, hierarchical clustering, or Gaussian mixture model may be utilized to categorize each segment of the physiological signals 305.

The output of the sleep classifier 415 or the sleep analyzer 405 is referred herein as a sleep architecture 420. The sleep architecture 420 may provide an overall organization of sleep, a broader structure, and a pattern of sleep across the monitored period (or the period of time), including the proportion of time spent in each stage and the progression through sleep-awake cycles. The sleep analyzer 405 may generate outputs that are included in the sleep architecture 420 such as a sleep score, preferred or dominant frequency analysis results, arousal detection results, microsleep detection results, landmark detection results, spectral or temporal fragmentation results, spectral or temporal signatures, or a hypnogram that categorizes sleep into stages such as awake, REM, and non-REM including, SWS, stage I, and stage II sleep. The sleep architecture 420 may also include the mean or maximum time between arousals or the fractions of arousals resulting in the overall quality of the sleep. By analyzing sleep quality, duration, and patterns, the sleep analyzer 405 may also compute metrics like the mean time between arousals.

According to some embodiments, the sleep architecture 420 may include a sleep score for each sleep-awake cycle or whole night sleep, intervals detected between arousals or micro-arousals, average sleep duration, and/or a hypnogram. The sleep score can be a numerical value indicating the quality of sleep and may consider factors like duration, depth, and consistency of sleep cycles. The arousal detection results may include instance records where the subject briefly wakes up or is disturbed during sleep, which can negatively impact sleep quality. The hypnogram can be a visual representation of sleep stages over time, categorizing sleep into various phases such as periods when the subject is fully awake, REM stage, and non-REM including, SWS, stage I, and stage II sleep. The hypnogram may further include microarousals, microsleeps, etc. In some instances, a single channel EEG data may be retrieved from the one or more database(s) 120 and can be used to perform the sleep stage analyses to generate the sleep architecture 420.

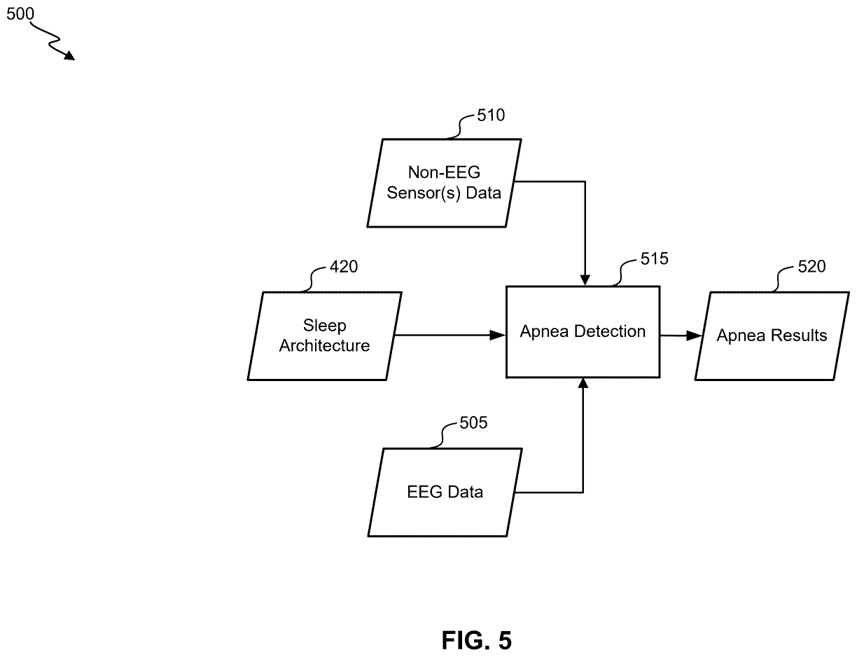

FIG. 5 illustrates an example implementation of an apnea detection 515 of the subject by leveraging output of the sleep analyzer 405 (or sleep architecture 420), EEG data 505, and one or more non-EEG sensor data 510. The EEG data 505 corresponds to EEG signals of the subject that were collected during sleeping state and were used by the sleep analyzer 405 to generate the sleep architecture 420. The arousal detection results can be retrieved from the sleep architecture 420 and may indicate brief awakenings or micro-arousal instants in the EEG data 505.

By utilizing the brief awakenings or micro-arousal instants as reference points, the apnea detection 515 may determine anomalies in the EEG signals around these points that can be indicative of apnea (e.g., OSA, or hypopnea). OSA often causes transient arousals, which can be detected as sudden changes in the EEG signals. Similarly, the apnea detection 515 may analyze changes in the EEG signals patterns that may occur during apneic episodes. For example, the EEG signals or brain activity might show brief bursts of high-frequency activity, changes in amplitude during apneic events, or removal of the one or more sleep stages.

In some embodiments, the one or more non-EEG sensor data 510 may be utilized by the apnea detection 515 to inform a detection of and/or severity estimation of an apneic event. For example, the non-EEG sensor data 510 may be processed in combination with the EEG data 505 using one or more models and/or processing techniques (e.g., such that an input data set includes one or more features or embeddings generated using the EEG data 505 and also one or more features or embeddings generated using the non-EEG data 510). As another example, the EEG data 505 and non-EEG sensor data 510 may be processed at different stages in a workflow (e.g., such that one or more features or embeddings generate using the EEG data 505 are first processed using a first model or technique to generate a predicted probability or severity of an apneic event and one or more features or embeddings generated using the non-EEG data 510 are then processed using a second model or technique to fine-tune, adjust and/or corroborate such predictions).

In some instances, the apnea detection 515 may generate the apnea risk based on the EEG data 505, the sleep architecture 420, or the one or more non-EEG sensor data 510. The one or more non-EEG sensor data 510 may include, for example, EKG sensors data, pulse oximeter data, audio data, video data, chest movement data, or nasal airflow data. The audio data may be collected using a microphone on the smartwatch, smartphone, tablet, laptop, and the like. Similarly, the video data can be collected using the user devices (e.g., smartphone, tablet, etc.). The one or more non-EEG sensor data 510 may be obtained in conjunction or synchronously with the EEG data 505. Pulse oximeter can measure blood oxygen levels, which is typically placed on a finger or earlobe. The pulse oximeter may help in identifying drops in oxygen saturation that can occur due to apneas or hypopneas. Moreover, periods of breathing cessation (apneas) or reduced airflow (hypopneas) may be identified through nasal airflow data. Further, the chest movement data can be used to assess respiratory effort and may be obtained using a chest effort belt that comprises sensors such as accelerometers, strain gauges, and the like. The apnea detection 515 may correlate the changes in the EEG data 505 at or around micro-arousals instants with the one or more non-EEG sensor data 510 (if available), such as pulse oximetry, heart rate, nasal airflow, chest movement. Thus, the apnea detection 515 may corroborate sleep disruptions due to respiratory disturbances.

In some instances, the apnea detection 515 may employ machine learning algorithms to classify and/or predict apnea events (e.g., OSA, hypopnea) based on EEG features. These algorithms can be trained on labeled datasets with known OSA events to learn patterns associated with the disorder (i.e., OSA).

After detecting the apneic events in the EEG data 505 corresponding to the period of time (e.g., a plurality of previous night-time periods), the apnea detection 515 may determine the duration and severity of each apneic event. The apnea detection 515 may further compute frequency of apneic events, mean severity, maximum severity, average duration, maximum duration, and the like. The frequency of apneic events may be computed per hour of sleep data (or EEG data 505), per each sleep-awake cycle, per each night, or during the period of time.

Apnea results 520 may include all the above computed or determined values by the apnea detection 515, for example, time stamps, severity, duration of apnea and hypopnea episodes. Moreover, the time stamps may be analyzed further by the apnea detection 515 to assess whether the apneic events occur randomly during the sleep-awake cycles, whether the events are more frequent during a specific sleep stage (e.g., SWS, REM sleep, etc.), or if they vary throughout the night. In addition, the apnea results may also include apnea-hypopnea index (AHI) that quantifies the number of apneas and hypopneas per hour of sleep. In some instances, based on one or more criteria such as if the frequency, duration, or severity of apneic events exceed a corresponding threshold value, the apnea detection 515 may screen the subject as having apnea or hypopnea, and may alert the subject or the caregiver. The AHI can help to classify the severity of OSA. The AHI value 5-15 events per hour may be considered as mild OSA, 15-30 events per hour as moderate OSA, and AHI values greater than 30 events per hour can be classified as severe OSA.

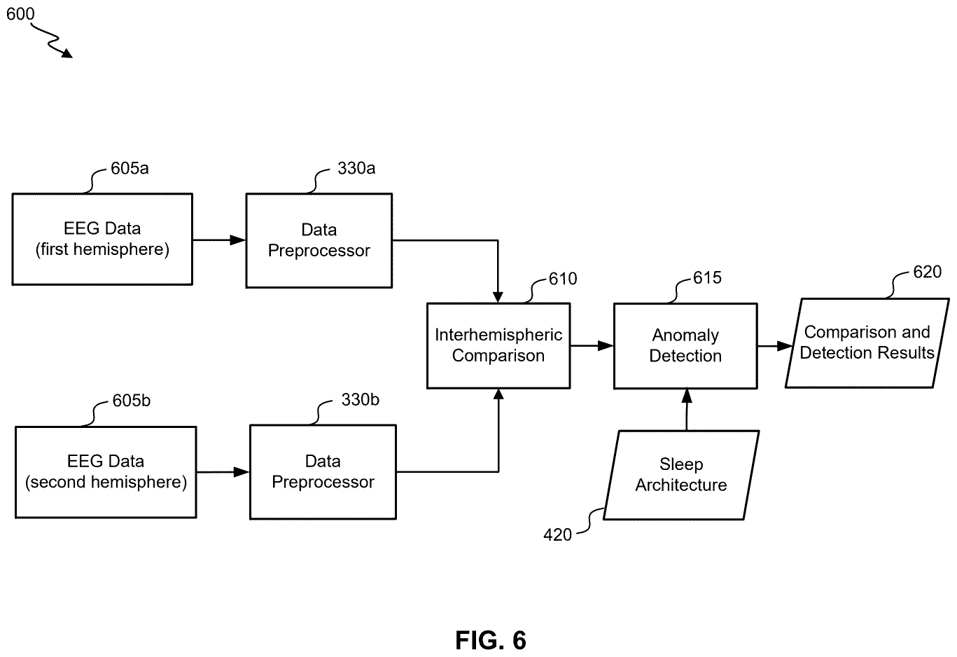

FIG. 6 shows an example illustration to process the EEG data 505 of the left and right hemisphere to detect stroke (or hemorrhagic stroke), mini-strokes, TIA, aneurysms, SBIs, brain tumor, or TBI in the brain of the subject. In some instances, by using the metadata, the EEG data 505 that may be retrieved from the one or more database(s) 120 can be separated based on the location of EEG electrodes on the head of the subject. EEG data of the first hemisphere 605a (e.g., left hemisphere) and EEG data of the second hemisphere 605b (e.g., right hemisphere) may be processed separately using a data preprocessor 330a and a data preprocessor 330b, respectively. Both data preprocessors 330a-b comprise of similar modules such as preprocessing 310, segmentation 315, transformation 320, and feature extraction 325.

EEG data (or signals) for each hemisphere 605a-605b may be processed by the data preprocessors 330a-b to remove noise and artifacts (e.g., muscle activity, eye movements) from the EEG signals. The EEG data for each hemisphere 605a-605b may be segmented into epochs or segments and can be transformed into frequency-domain. In some instances, one or more frequency bands (e.g. Delta, Theta, Alpha, Beta, Gamma) bands may be identified and the set of features as described in FIG. 3 can be extracted from the EEG signals.

Afterwards, an interhemispheric comparison 610 may be performed on the EEG data of the first hemisphere 605a and the EEG data of the second hemisphere 605b to analyze or quantify asymmetry across hemispheres that may be indicative of a neurodegenerative abnormality (e.g., SBI, TIA, stroke, TBI, or tumor). In some embodiments, for the interhemispheric comparison 610, coherence may be computed across hemispheres to exploit bilateral symmetry in brain activity during sleeping or resting state. Coherence may provide valuable insights into the functional connectivity and synchronization of brain activity across different regions and hemispheres. Coherence values range from 0 to 1. The ‘0 ’ indicates no coherence or no synchronization between the two EEG signals (e.g., EEG data of the first hemisphere 605a and EEG data of the second hemisphere 605b), and the signals are entirely independent of each other. Coherence values close to ‘0’ or low coherence may reflect reduced interhemispheric connectivity, communication, or lack of synchronization between the two EEG signals. Similarly, the value ‘1’ indicates ideal coherence and may refer to situations when the signals are completely synchronized with each other and have the same frequency and phase relationship. The high coherence values (close to 1) are indicative of effective interhemispheric communication, coordination, or well synchronization between the two hemispheres.

During resting or sleeping states, the EEG data 505 or signals exhibits bilateral symmetry. The neurodegenerative abnormalities usually are unilateral, hence, a decay or deterioration in coherence across hemispheres may indicate stroke, mini-stroke, TIA, SBI, brain tumor, or TBI. The coherence across hemispheres may be computed for each segment or epoch of the EEG data (or signals) for each hemisphere 605a-605b, or even at each frequency for each time point. In some instances, by utilizing the output of the sleep analyzer 405 (e.g., the sleep architecture 420), coherence across hemispheres may be computed by using the EEG data (or signals) for each hemisphere 605a-605b corresponding to intervals such as sleep-awake cycles, or different sleep states. In some examples, coherence may also be computed in different frequency bands (e.g., Delta, Theta, Alpha, Beta, Gamma) to reveal different aspects of brain activity. The coherence may be computed based on the techniques but are not limited to magnitude-squared coherence (MSC), cross-spectral density (CSD) (e.g., for general coherence analysis), short-time Fourier transform (STFT), and wavelet transform (e.g., for analyzing coherence over time and frequency).

In some other embodiments, for the interhemispheric comparison 610, a plurality of the set of features (or feature sets) may be used that are extracted for each time interval, segment, or epoch of the EEG signals and for each of the two hemispheres by using the data preprocessors 330a-b. These feature sets may represent characteristics of neural activity over time and can be used to quantify differences between the right and the left hemispheres.

In some instances, to analyze asymmetry or abnormality in neural activity between the two hemispheres of the brain, a multi-dimensional distribution may be generated by using the feature sets corresponding to each hemisphere. Statistical analysis may be performed on the multi-dimensional distributions to determine whether they stem from the same underlying distribution or if there are significant differences indicative of neurodegenerative abnormalities. The statistical analysis may utilize one or more statistical tests such as Kolmogorov-Smirnov test, t-test or Wilcoxon signed-rank test. The statistical tests may yield a p-value that quantifies the probability that observed differences between the distributions are due to chance. A smaller value of p-value (e.g., p-value less than 0.05) may be indicative of asymmetry in neural activity across hemispheres and presence of neurodegenerative abnormality.

In some other instances, a clustering technique may be applied for determination of asymmetry between the EEG data of the first hemisphere 605a and the EEG data of the second hemisphere 605b. The clustering techniques may include but are not limited to k-means clustering, hierarchical clustering or Gaussian mixture model (GMM). Each feature set (either representing EEG data of the first hemisphere 605a or the second hemisphere 605b) corresponding to a given epoch and a given hemisphere may be assigned to a cluster. Clustering may group similar feature vectors together, thereby identifying patterns and similarities within and between the first and the second hemispheres. After the clustering process, a quantification of how closely related or different the neural activity patterns are between the two hemispheres can be performed using the clusters or clustering results. A probabilistic measure or metric of asymmetry can be computed by comparing the distribution of feature vectors assigned to clusters in the first hemisphere with those in the second hemisphere. This comparison may involve statistical metrics such as the Kolmogorov-Smirnov test, which evaluates differences in the distributions of feature vectors. To quantify clusters asymmetry, a probability value or score can be derived based on how dissimilar the clusters are between the left (e.g., first) and right (e.g., second) hemispheres. Higher dissimilarity across clusters of the two hemispheres may be reflected by lower similarity scores or higher probability values (indicating significant differences in distributions). The probability value may correspond to the neurodegenerative abnormality.

In some other instances, the feature sets or the EEG data of the first hemisphere 605a and the second hemisphere 605b may then be projected into a multi-dimensional space, where each point represents a feature vector from either the left or the right hemisphere. For interhemispheric comparison 610, one or more inter-hemisphere distance metrics may be computed based on the distances between feature vectors from different hemispheres. The one or more inter-hemisphere distance metrics can be obtained by calculating various distance measures, such as Euclidean distance, Mahalanobis distance (covariance between features), or cosine similarity (measure angular difference). These metrics capture the dissimilarity or similarity in feature vectors between corresponding points across hemispheres. Moreover, one or more intra-hemisphere distance metrics can also be computed within each hemisphere, reflecting distances between feature vectors from the same hemisphere. These intra-hemisphere metrics provide a baseline of similarity for comparison against inter-hemisphere distances.

A composite score may be formulated using these distance metrics. One approach is to define a score that incorporates both inter-hemisphere distances (reflecting asymmetry) and intra-hemisphere distances (reflecting bilateral similarity). For instance, a ratio or difference between inter-hemisphere and intra-hemisphere distances can quantify the degree of asymmetry, where larger ratios or differences suggesting greater asymmetry between hemispheres and a higher risk of neurodegenerative abnormality. Additionally, statistical techniques such as computing z-scores or normalized distances relative to a reference distribution (e.g., healthy controls) can further refine the assessment of asymmetry. The statistical normalization can account for individual variability and can increase the sensitivity of detecting abnormal asymmetry patterns indicative of neurodegenerative conditions.

An anomaly detection 615 may be utilized to analyze the results of the interhemispheric comparison 610, for example, coherence patterns, the p-value, the probability, or the score and to detect a presence of the neurodegenerative abnormality in the brain of the subject. For example, the p-value that is generated using the statistical analysis can be compared to a p-value threshold. A smaller value of p-value (e.g., p-value less than 0.05) may be indicative of asymmetry in neural activity across hemispheres and presence of neurodegenerative abnormality. Moreover, by comparing the composite score against a predefined threshold, the anomaly detection 615 can determine the presence and severity of asymmetry in neural activity between brain hemispheres. Furthermore, the probability value (e.g., derived from clusters analysis) can then be compared to a predefined probability threshold. The probability value greater than the probability threshold, indicates neural dysfunction or pathology. In the case of healthy subjects (i.e., without neurodegenerative abnormalities) the clustering may reveal that the feature sets extracted from the EEG signals of the left and right hemispheres form cohesive clusters, indicating bilateral symmetry in neural activity patterns.

After detecting the presence of the neurodegenerative abnormality, the anomaly detection 615 may further detects or identify the presence or likelihood of the hemorrhagic stroke, mini-stroke, TIA, SBIs, aneurysms, brain tumor, or TBI, based on the apnea, the apnea risk, the change in sleep, and the interhemispheric comparison 610 results (e.g., coherence, p-value, probability value, score, etc.). In some embodiments, the anomaly detection 615 may compare the coherence values or patterns, for example, during different sleep-awake cycles, different sleep stages, time derivatives, or patterns, with baseline data of control groups or healthy individuals. The anomaly detection 615 may utilize the sleep architecture 420 to retrieve the one or more biomarkers such as the indication of apnea, the apnea risk, the change in sleep (e.g., reduced SWS), and the like. The anomaly detection 615 may utilize the sleep architecture 420 and the interhemispheric comparison results to discriminate different neurodegenerative abnormalities. For instance, an occlusion in the brain can change the coherence. A tumor usually slows down the EEG locally, thereby disproportionately affecting coherence at higher frequencies. Moreover, TBI can disrupt sleep patterns, often leading to reduced time in SWS (Stage 3) and alterations in Stage 2 sleep. If none of these disturbances are seen (e.g., tumor and TBI associated disturbances), and if on top of that, there is apnea and SWS suppression is present, and the subject does not show impairment, then the anomaly detection 615 may identify or detect an aneurysm or SBI. If the subject exhibit impairments, then it is likely mini-stroke, TIA, or stroke. The anomaly detection 615 may also perform longitudinal analysis on the subject past or historical data to identify each of the neurodegenerative abnormalities such as stroke, mini-strokes, TIA, SBIs, brain tumor, or TBI.

In some other embodiments, the EEG data 505 (or EEG data of the first hemisphere 605a and EEG data of the second hemisphere 605b) may be collected from a plurality of subjects in a prospective study design. At baseline, the plurality of subjects may represent those individuals who are at risk of stroke and have no history of stroke, mini-stroke, TIA, or SBI. The subjects that have three or more stroke risk factors on the stroke risk score card may be considered at risk of stroke and may be selected. The stoke risk factors on the stroke risk score card may include: blood pressure greater than 120/80 mm/Hg; diagnosed with atrial fibrillation; blood sugar greater than 100 mg/dL; BMI greater than 25 kg/m²; diet high in saturated fat, trans fat, sweetened beverages, salt, excess calories; total blood cholesterol greater than 160 mg/dL; diagnosed with diabetes mellitus; less than 150 minutes of moderate to vigorous intensity activity per week; smoking; between 40-75 years of age; without a diagnosis of dementia or cognitive impairment hindering participation in the prospective study; and family history of stroke, TIA, or heart attack.

The EEG data 505 and neuroimaging test (e.g., MRI, CT) may be collected at baseline and at one or more future time intervals. Neuroimaging test results may be considered ground truth to classify or categorize individuals (or the EEG data 505) into two or more groups such as healthy individuals (or control groups), and impaired individuals. The impaired individual may further be categorized into individuals with stroke, individuals with mini-strokes (or TIA), and individuals with SBI. The interhemispheric comparison 610 results, the apnea results 520, and the sleep architecture 420 may be computed for each of these groups using the corresponding EEG signals and may be used to train a machine learning model. The anomaly detection 615 may utilize the trained machine learning model to screen for individuals who may have had a SBI or a mini-stroke in recent past. The machine learning model may include but not limited to deep learning models, transformer models, decision tree (DT), random forest (RF), support vector machines (SVM), neural networks (NNs), and the Like.

Further, the anomaly detection 615 may output comparison and detection results 620. The comparison and detection results 620 may include values indicative of presence, likelihood, or severity of stroke, mini-stroke, TIA, SBIs, aneurysms, brain tumor, or TBI. In addition, the interhemispheric comparison 610 results may also be included in the comparison and detection results 620 for further analysis, for example, by the clinician. In some instances, one or more biomarkers based on the sleep architecture 420 or the apnea results 520 may also be included in the comparison and detection results 620.

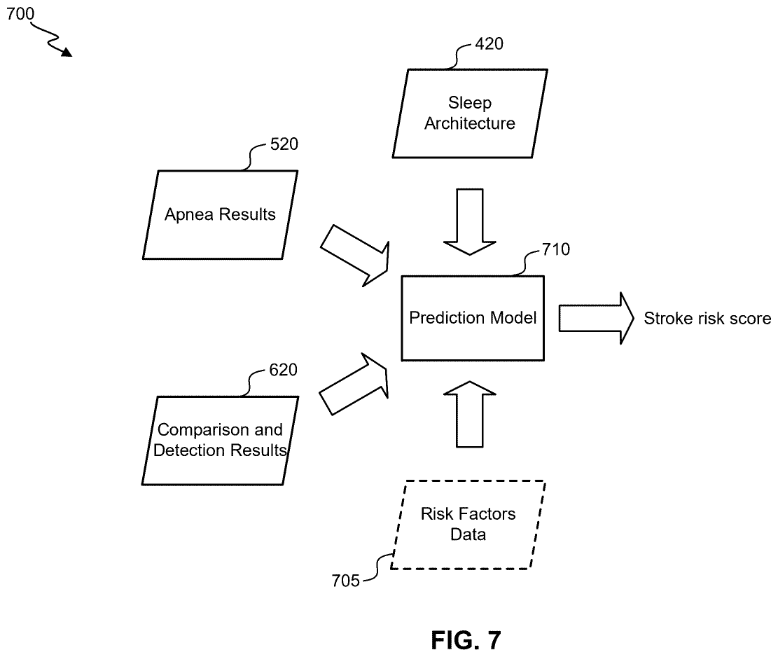

FIG. 7 illustrates an example architecture to generate a stroke risk score of the subject by using a prediction model 710. The prediction model 710 may leverage the sleep architecture 420, the apnea results 520, the comparison and detection results 620, and the risk factors data 705 of the subject to predict the stroke risk score. The sleep architecture 420, the apnea results 520, the comparison and detection results 620, can be obtained based on the analysis of the physiological signals 305 (or EEG data 505) of the subject. One or more biomarkers may be identified from the sleep architecture 420, the apnea results 520, the comparison and detection results 620, and risk factors data (e.g., other risk factors or non-EEG based risk factors). The one or more biomarkers may include an indication of apnea, or an indication of SBI. The one or more biomarkers further include a presence of OSA, a duration of OSA, an intensity of OSA, or a reduction in duration of SWS, a reduction in duration of REM, a presence of one or more SBI, a detection of TIA, a reduction in coherence across hemispheres, and the like.

The risk factors data 705 may be obtained with consent from the subject or a guardian from an electronic health record (EHR) that includes historical medical information of the subject. The risk factors data 705 may further be obtained, with consent from the subject or a guardian, from the subject's device such as smartwatch, smartphone that logs activities of daily life of the subject. The risk factors data 705 may be comprised of medical information and mobility metrics of the subject. The medical information may include but is not limited to a BMI value, an atrial fibrillation diagnosis, a diabetes mellitus diagnosis, a blood sugar level history, a blood pressure data, a family history of stroke or TIA, a family history of heart attack, or a blood cholesterol level. The mobility metrics may include, for example, an intensity of physical activity per week.

The prediction model 710 may be trained on a dataset that can be generated using a prospective study design as explained in FIG. 6. The physiological signals 305 and neuroimaging data (e.g., MRI, CT) may be collected at baseline and at one or more future time intervals. The data about future stroke events (including mini-strokes, TIA, SBI) may also be collected and those individuals may be categorized into impaired group or high-risk group. In some instances, multiple groups can be defined based on the risk factors, neuroimaging data, and clinical assessments. Afterwards, the physiological signals 305 may be analyzed by the techniques as disclosed in the present disclosure to obtain one or more biomarkers. After training, the prediction model 710 may be utilized to generate stroke risk score of a subject preferably in home settings for early detection of stroke. In some instances, the prediction model 710 may generate the stroke risk score based on the one or more biomarkers (e.g., duration, severity, frequency of the biomarkers, etc.). In some other instances, risk factors data 705 (if available) can be used together with the one or more biomarkers to predict stroke risk score of the subject. In other instances, the disclosed techniques can be used to assess an efficacy of interventions or to monitor recovery of a subject who had experienced a neurodegenerative abnormality such as a stroke.

The prediction model 710 can include a machine-learning model, such as a recurrent neural network (RNN), a long short-term memory (LSTM) model, a transformer model, neural networks (NNs), multi-value prediction algorithm, deep learning models, or regression techniques. The stroke risk score indicates a likelihood or a probability that the subject will experience a stroke or mini-stroke in a future time period (e.g., in next 1 week, month, few months, or year). The stroke risk score may include, for example, a number (e.g., an integer or real number selected from a defined scale such as 0-10) or a category. The categories can be mild, moderate, or high risk. The stroke risk score can be binary in some cases and may discriminate or screen individuals at risk of stroke.

In addition, one or more actions can be performed by the neurodegenerative abnormality detection system based on the stroke risk score of the subject. The one or more actions may include, but not limited to, generating alerts, notifying the concerned authorities (e.g., medical staff, relatives, and/or the subject) for a complete medical evaluation to confirm the occurrence or risk of stroke, mini-stroke, TIA, or the presence of one or more SBIs or aneurysms.

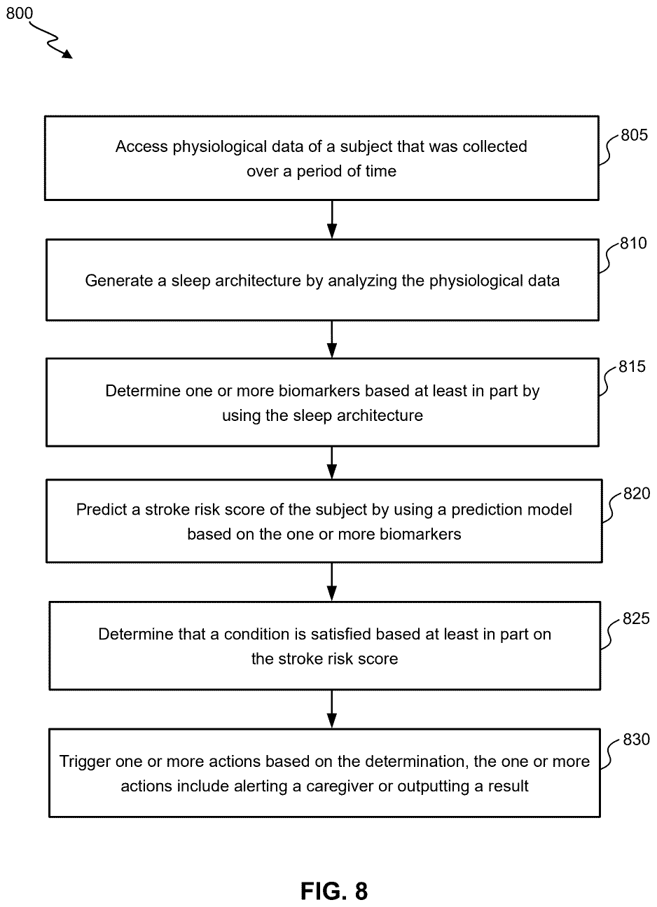

FIG. 8 shows an example flowchart of a system for detecting risk or occurrence of stroke or infarction in a subject in accordance with some embodiments of the present disclosure. The blocks in flowchart 800 are illustrated in a specific order, while the order can be modified, for example, some blocks may be performed before other, and some blocks may be performed simultaneously. The blocks can be performed by hardware or software or a combination thereof. The process at block 805 may include accessing physiological data of the subject that was collected by the physiological data acquisition assembly over the period of time. The physiological data may correspond to physiological signals (e.g., EEG, EMG, EOG, EKG, MEG) that were collected during a night-time period, or a plurality of previous night-time periods. The physiological data acquisition assembly may be comprised of the sensing device and the one or more clusters of electrodes. Each cluster of the one or more clusters of electrodes comprises at least an active electrode. In some instances, the electrodes can be EEG electrodes to record the EEG signals of the left and right hemisphere of the brain of the subject.

The sleep architecture 420 may be generated by analyzing the physiological data of the subject, at block 810. In some instances, to generate the sleep architecture 420, the set of features may be extracted at first based on a portion of the physiological data corresponding to each time interval of a plurality of time intervals within the period of time. A state can be predicted for each of the set of features corresponding to each time interval. The state may correspond to any of one or more sleep stages or an awake state. The one or more sleep stages may include REM stage, and the one or more non-REM stages (including the SWS stage). Based on the predicted states for each time interval, the sleep pattern, the relative frequency, and the duration of each of the one or more sleep or awake states (e.g., quiet wakefulness and active wakefulness) can be determined.

One or more biomarkers may be determined based at least in part by using the sleep architecture 420 of the subject, at block 815. In some instances, one or more biomarkers may further be determined based on the first portion and the second portion of the physiological data such as EEG data 505 corresponding to the left hemisphere and the right hemisphere of the subject, respectively. Afterwards, the coherence can be computed between the first portion and the second portion of the physiological data. A presence or likelihood of the hemorrhagic stroke, SBIs, TIA, aneurysm, brain tumor, or TBI may be determined based on the coherence, the apnea, the apnea risk, and the change in sleep. Moreover, the one or more biomarkers may further include a presence of obstructive sleep apnea (OSA), a duration of OSA, an intensity of OSA, or a reduction in duration of slow wave sleep (SWS).

The stroke risk score of the subject may be predicted using the prediction model 710 based on the one or more biomarkers, at block 820. In some embodiments, the prediction model 710 may leverage the risk factors data 705 (non-EEG based), in addition to the one or more biomarkers, to generate the stroke risk score. The risk factors data 705 may be comprised of medical information and the mobility metrics of the subject. The prediction model 710 may be comprised of a machine learning model such as the RNN model, the transformer model, the deep learning models, or the neural network.

The process at block 825 may determine that a condition is satisfied based at least in part on the stroke risk score. For example, the condition can be such that whether the (predicted) stroke risk of the subject is greater than a specific threshold, whether the stroke risk score is indicative of high risk of future stroke, or whether the stroke risk score is medium along with either apnea is present or SBI (including mini-stroke or TIA) is detected, and the like.

Finally, at block 830, the one or more actions may be triggered based on determining that the condition is satisfied. The one or more actions may include alerting the subject, alerting a caregiver or a clinician, or outputting a result or data that provides a basis for a recommendation or includes the recommendation to perform an evaluation or an intervening action to reduce the stroke risk score. The one or more actions may further include presenting the result and/or the stroke risk score of the subject on the computing device 115 or transmitting the result to another device. The outputting the result or the data may further include the sleep architecture 420, the apnea results 520, and the comparison and detection results 620 to facilitate the clinician, whether human or not, for further investigation and thence for the relevant clinician to recommend a treatment plan for the subject.

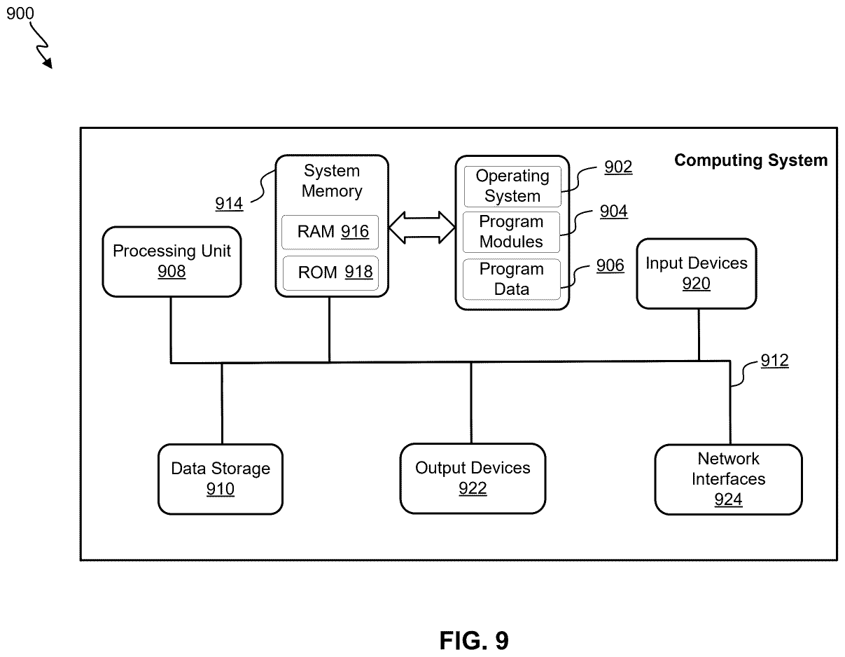

FIG. 9 shows an example illustration of the computing system 900 in which various embodiments of the present disclosure may be implemented. The computing system 900 can be used as the computing device 115 as explained in FIG. 1. For example, the techniques as disclosed above in the present disclosure for detecting the risk or occurrence of stroke using the physiological data (or EEG signals) can be implemented in computer-executable instructions (e.g., organized in program modules 904). The program modules 904 can include the routines, programs, objects, components, and data structures that perform the tasks and implement the data types for implementing the techniques described above. The functionality described herein can be performed, at least in part, by one or more hardware logic components.

To provide additional context for various aspects thereof, FIG. 9 and the following description are intended to provide a brief, general description of the computing system 900 in which the various aspects can be implemented. While the description above is in the general context of computer-executable instructions that can run on one or more computers, those skilled in the art will recognize that a novel implementation also can be realized in combination with other program modules and/or as a combination of hardware and software. Computing system 900 or computer system for implementing various aspects includes a processing unit 908 having one or more processors (also referred to as microprocessors), a computer-readable storage medium (where the medium is any physical device or material on which data can be electronically and/or optically stored and retrieved) such as a data storage 910 unit (computer-readable storage medium/media also include magnetic disks, optical disks, solid state drives, external memory systems, and flash memory drives), and a system bus 912. The system bus 912 may provide an interface for system components including, but not limited to, system memory 914, to the processing unit 908. Such a system bus 912 can be of any of several types of bus structure that can further interconnect to memory bus (with or without controller), and a peripheral bus (e.g., Peripheral Component Interconnect (PCI), Peripheral Component Interconnect Express (PCIe), Accelerated Graphics Port (AGP), Low Pin Count (LPC), etc.), using any of a variety of commercially available bus architectures.

FIG. 9 shows an example configuration of a typical computer that may be other commercially available microprocessors such as single-processor, multi-processor, single-core units, and multi-core units of processing and/or storage circuits. Moreover, those skilled in the art will appreciate that the novel system and methods can be practiced with other computer system configurations, including minicomputers, mainframe computers, as well as personal computers (e.g., desktop, laptop, tablet PC, etc.), hand-held computing devices, microprocessor-based or programmable consumer electronics, and the like, each of which can be cooperatively coupled to one or more associated devices.

In some aspects, the computing system 900 can be one of several computers employed in a datacenter and/or computing resources (hardware and/or software) in support of cloud computing services for portable and/or mobile computing systems such as wireless communications devices, cellular telephones, and other mobile-capable devices. Cloud computing services, include, but are not limited to, infrastructure as a service, platform as a service, software as a service, storage as a service, desktop as a service, data as a service, security as a service and APIs (application program interlaces) as a service, for example. In some instances, the system memory 914 can include computer-readable storage (physical storage) medium such as a volatile memory (e.g. random-access memory (RAM) 916) and a non-volatile memory (e.g., read only memory (ROM) 918). A basic Input/output system (BIOS) can be stored in the non-volatile memory and includes the basic routines that facilitate the communication of data and signals between components within the computing system 900, such as during startup. The volatile memory also includes a high-speed RAM such as static RAM for caching data.

By way of example, and not limitation, the system memory 914 also may also include program modules 904, which may include client applications, Web browsers, mid-tier applications, relational database management systems (RDBMS), etc., program data 906, and an operating system 902. By way of example, operating system 902 may include various versions of Microsoft Windows®, Apple Macintosh®, and/or Linux operating systems, a variety of commercially-available UNIX® or UNIX-like operating systems (OS) (including without limitation the variety of Gnu's Not Unix (GNU)/Linux operating systems, the Google Chrome OS, and the like) and/or mobile operating systems such as iOS, Windows® Phone, Android OS, BlackBerry® OS, and Palm® OS operating systems. All or portions of operating system 902, program modules 904, and/or program data 906 can also be cached in memory such as the volatile memory and/or non-volatile memory, for example (RAM 916 or ROM 918). It is to be appreciated that the disclosed architecture can be implemented with various commercially available operating systems or combinations of operating systems (e.g., virtual machines).

In some other examples, the computing system 900 may have additional features or functionality. For example, the computing system 900 may also include additional data storage devices (removable and/or non-removable) such as, for example, magnetic disks, optical disks, or tape. Computer-readable media may include, at least, two types of computer-readable media, namely computer storage media and communication media. Computer storage media may include volatile and non-volatile, removable, and non-removable media implemented in any method or technology for storage of information, such as computer-readable instructions, data structures, program modules, or other data.

The system memory 914, and the data storage 910 including removable storage, and non-removable storage are all examples of computer storage media. Apart from RAM 916 and ROM 918, computer storage media includes, but is not limited to, electrically erasable programmable ROM (EEPROM), flash memory or other memory technology, compact disc (CD)-ROM, digital versatile disks (DVD), or other optical storage, magnetic cassettes, magnetic tape, magnetic disk storage or other magnetic storage devices, or any other non-transmission medium that can be used to store the targeted information and which can be accessed by the computing system 900. Moreover, the computer-readable media may include computer-executable instructions that, when executed by the processing unit 908, perform various functions and/or operations described herein. The communication media may embody computer-readable instructions, data structures, program modules, or other data in a modulated data signal, such as a carrier wave, or other transmission mechanism.

The computing system 900 may also include one or more input devices 920 such as keyboard, mouse, pen, voice input device, touch input device, etc. One or more output devices 922 such as a display, speakers, printers, etc. may also be included. These devices are well known in the art and are not discussed at length here. The computing system 900 may also include one or more network interfaces 924 to establish communication that may allow computing system 900 to communicate with other system or devices, such as over a network. These networks may include wired networks as well as wireless networks. Here, the computing system 900 is one example of a suitable device or system and is not intended to suggest any limitation as to the scope of use or functionality of the various embodiments described.

Other well-known computer systems, environments and/or configurations that may be suitable for use with the embodiments include, but are not limited to personal computers, server computers, hand-held or laptop devices, multiprocessor systems, microprocessor-based systems, set top boxes, game consoles, programmable consumer electronics, network PCs, minicomputers, mainframe computers, distributed computing environments that include any of the above systems or devices, and/or the like. For example, some or all of the components of computing system 900 may be implemented in a cloud computing environment, such that resources and/or services are made available via a computer network for selective use by the user devices.

Some embodiments of the present disclosure include a system including one or more data processors. In some embodiments, the system includes a non-transitory computer-readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A computer-implemented method comprising:
accessing physiological data of a subject that was collected over a period of time by a physiological data acquisition assembly, wherein the physiological data acquisition assembly comprises a sensing device and one or more clusters of electrodes, and wherein each cluster of the one or more clusters of electrodes comprises at least an active electrode;
generating a sleep architecture by analyzing the physiological data;
determining one or more biomarkers based at least in part by using the sleep architecture, wherein the one or more biomarkers include an indication of apnea, an apnea risk, a change in sleep, or a change across hemispheres;
predicting a stroke risk score of the subject by using a prediction model based on the one or more biomarkers;
determining that a condition is satisfied based at least in part on the stroke risk score; and
triggering one or more actions based on determining that the condition is satisfied, wherein the one or more actions include alerting the subject, alerting a caregiver or a clinician, or outputting a result that provides a basis for a recommendation or includes the recommendation to perform an evaluation or an intervening action to reduce the stroke risk score.

2. The computer-implemented method of claim 1, wherein generating the sleep architecture includes:
extracting, for each time interval of a plurality of time intervals within the period of time, a set of features based on a portion of the physiological data;
predicting a state for each of the set of features corresponding to each time interval, wherein the state corresponds to any of one or more sleep stages or an awake state, and wherein the one or more sleep stages include a rapid eye movement (REM) stage and one or more non-REM stages; and
determining, based on predicting the state:
a sleep pattern;
a relative frequency of each of the one or more sleep stages or the awake state; and
a duration of each of the one or more sleep stages or the awake state.

3. The computer-implemented method of claim 1, wherein determining the one or more biomarkers further comprising:
determining a first portion of the physiological data corresponding to a first hemisphere of a brain of the subject;
determining a second portion of the physiological data corresponding to a second hemisphere of the brain of the subject;
computing a coherence between the first portion and the second portion of the physiological data; and
determining, based on the apnea, the apnea risk, the change in sleep, and the coherence, a presence or likelihood of a hemorrhagic stroke, silent brain infarcts (SBI), a transient ischemic stroke (TIA), an aneurysm, a brain tumor, or a traumatic brain injury (TBI).

4. The computer-implemented method of claim 1, wherein the one or more biomarkers further include a presence of obstructive sleep apnea (OSA), a duration of OSA, an intensity of OSA, or a reduction in duration of slow wave sleep (SWS), a presence of SBI, or a detection of TIA.

5. The computer-implemented method of claim 1, wherein the stroke risk score is further predicted based on risk factors data comprising medical information and mobility metrics of the subject, wherein the medical information includes a body mass index (BMI) value, an atrial fibrillation diagnosis, a diabetes mellitus diagnosis, a blood sugar level history, a blood pressure data, a family history of stroke or TIA, a family history of heart attack, or a blood cholesterol level, and wherein the mobility metrics include an intensity of physical activity per week.

6. The computer-implemented method of claim 1, wherein the physiological data corresponds to physiological signals that were collected during a night-time period, a rest period, or a plurality of previous night-time or rest periods.

7. The computer-implemented method of claim 1, wherein the prediction model comprises of a machine learning model including a recurrent neural network (RNN), a transformer model, or a neural network.

8. The computer-implemented method of claim 1, wherein the one or more clusters of electrodes include electroencephalogram (EEG) electrodes, electromyography (EMG) electrodes, magnetoencephalography (MEG) electrodes, or electrooculogram (EOG) electrodes.

9. A system comprising:
one or more data processors; and
a non-transitory computer-readable storage medium containing instructions which, when executed on the one or more data processors, cause the one or more data processors to perform a set of operations including:
accessing physiological data of a subject that was collected over a period of time by a physiological data acquisition assembly, wherein the physiological data acquisition assembly comprises a sensing device and one or more clusters of electrodes, and wherein each cluster of the one or more clusters of electrodes comprises at least an active electrode;
generating a sleep architecture by analyzing the physiological data;
determining one or more biomarkers based at least in part by using the sleep architecture, wherein the one or more biomarkers include an indication of apnea, an apnea risk, a change in sleep, or a change across hemispheres;
predicting a stroke risk score of the subject by using a prediction model based on the one or more biomarkers;
determining that a condition is satisfied based at least in part on the stroke risk score; and
triggering one or more actions based on determining that the condition is satisfied, wherein the one or more actions include alerting the subject, alerting a caregiver or a clinician, or outputting a result that provides a basis for a recommendation or includes the recommendation to perform an evaluation or an intervening action to reduce the stroke risk score.

10. The system of claim 9, wherein generating the sleep architecture includes:
extracting, for each time interval of a plurality of time intervals within the period of time, a set of features based on a portion of the physiological data;
predicting a state for each of the set of features corresponding to each time interval, wherein the state corresponds to any of one or more sleep stages or an awake state, and wherein the one or more sleep stages include a rapid eye movement (REM) stage and one or more non-REM stages; and
determining, based on predicting the state:
a sleep pattern;
a relative frequency of each of the one or more sleep stages or the awake state; and
a duration of each of the one or more sleep stages or the awake state.

11. The system of claim 9, wherein determining the one or more biomarkers further comprising:
determining a first portion of the physiological data corresponding to a first hemisphere of a brain of the subject;
determining a second portion of the physiological data corresponding to a second hemisphere of the brain of the subject;
computing a coherence between the first portion and the second portion of the physiological data; and
determining, based on the apnea, the apnea risk, the change in sleep, and the coherence, a presence or likelihood of a hemorrhagic stroke, silent brain infarcts (SBI), a transient ischemic stroke (TIA), an aneurysm, a brain tumor, or a traumatic brain injury (TBI).

12. The system of claim 9, wherein the one or more biomarkers further include a presence of obstructive sleep apnea (OSA), a duration of OSA, an intensity of OSA, or a reduction in duration of slow wave sleep (SWS), a presence of SBI, or a detection of TIA.

13. The system of claim 10, wherein the stroke risk score is further predicted based on risk factors data comprising medical information and mobility metrics of the subject, wherein the medical information includes a body mass index (BMI) value, an atrial fibrillation diagnosis, a diabetes mellitus diagnosis, a blood sugar level history, a blood pressure data, a family history of stroke or TIA, a family history of heart attack, or a blood cholesterol level, and wherein the mobility metrics include an intensity of physical activity per week.

14. The system of claim 9, wherein the physiological data corresponds to physiological signals that were collected during a night-time period, a rest period, or a plurality of previous night-time or rest periods.

15. The system of claim 9, wherein the prediction model comprises of a machine learning model including a recurrent neural network (RNN), a transformer model, or a neural network.

16. A computer-program product tangibly embodied in a non-transitory machine-readable storage medium, including instructions configured to cause one or more data processors to perform a set of operations comprising:
accessing physiological data of a subject that was collected over a period of time by a physiological data acquisition assembly, wherein the physiological data acquisition assembly comprises a sensing device and one or more clusters of electrodes, and wherein each cluster of the one or more clusters of electrodes comprises at least an active electrode;
generating a sleep architecture by analyzing the physiological data;
determining one or more biomarkers based at least in part by using the sleep architecture, wherein the one or more biomarkers include an indication of apnea, an apnea risk, a change in sleep, or a change across hemispheres;
predicting a stroke risk score of the subject by using a prediction model based on the one or more biomarkers;
determining that a condition is satisfied based at least in part on the stroke risk score; and
triggering one or more actions based on determining that the condition is satisfied, wherein the one or more actions include alerting the subject, alerting a caregiver or a clinician, or outputting a result that provides a basis for a recommendation or includes the recommendation to perform an evaluation or an intervening action to reduce the stroke risk score.

17. The computer-program product of claim 16, wherein generating the sleep architecture includes:
extracting, for each time interval of a plurality of time intervals within the period of time, a set of features based on a portion of the physiological data;
predicting a state for each of the set of features corresponding to each time interval, wherein the state corresponds to any of one or more sleep stages or an awake state, and wherein the one or more sleep stages include a rapid eye movement (REM) stage and one or more non-REM stages; and
determining, based on predicting the state:
a sleep pattern;
a relative frequency of each of the one or more sleep stages or the awake state; and
a duration of each of the one or more sleep stages or the awake state.

18. The computer-program product of claim 16, wherein determining the one or more biomarkers further comprising:
determining a first portion of the physiological data corresponding to a first hemisphere of a brain of the subject;
determining a second portion of the physiological data corresponding to a second hemisphere of the brain of the subject;
computing a coherence between the first portion and the second portion of the physiological data; and
determining, based on the apnea, the apnea risk, the change in sleep, and the coherence, a presence or likelihood of a hemorrhagic stroke, silent brain infarcts (SBI), a transient ischemic stroke (TIA), an aneurysm, a brain tumor, or a traumatic brain injury (TBI).

19. The computer-program product of claim 16, wherein the one or more biomarkers further include a presence of obstructive sleep apnea (OSA), a duration of OSA, an intensity of OSA, or a reduction in duration of slow wave sleep (SWS), a presence of SBI, or a detection of TIA.

20. The computer-program product of claim 16, wherein the stroke risk score is further predicted based on risk factors data comprising medical information and mobility metrics of the subject, wherein the medical information includes a body mass index (BMI) value, an atrial fibrillation diagnosis, a diabetes mellitus diagnosis, a blood sugar level history, a blood pressure data, a family history of stroke or TIA, a family history of heart attack, or a blood cholesterol level, and wherein the mobility metrics include an intensity of physical activity per week.
